(19) United States
(12) Patent Application Publication
(10) Pub. No.: US 2018/0148787 A1
(43) Pub. Date: May 31, 2018

(54) METHOD FOR DETERMINING THE RISK OF BRONCHOPULMONARY DYSPLASIA OF PRETERM INFANTS

(71) Applicant: Meribank Biotech Co., Ltd., New Taipei City (TW)

(72) Inventors: Chang-Yo Hsuan, Taipei City (TW); Willie Lin, Taipei City (TW); Wei-Ting Liu, Taipei City (TW)

(73) Assignee: Meribank Biotech Co., Ltd., New Taipei City (TW)

(21) Appl. No.: 15/787,808

(22) Filed: Oct. 19, 2017

Related U.S. Application Data
(60) Provisional application No. 62/426,861, filed on Nov. 28, 2016.

(51) Int. Cl. C12Q 1/68

(52) U.S. Cl. CPC C12Q 1/6883; C12Q 2600/118; C12Q 2600/158

(57) ABSTRACT

Provided is a method for determining the risk of bronchopulmonary dysplasia (BPD) of a preterm infant. The method comprises detecting the expression level of EPHB2 in a primary culture of mesenchymal stem or stromal cells (MSCs) derived from a placenta-related tissue of the infant's mother or umbilical cord blood of the infant.

### FIELD OF THE INVENTION

The present invention pertains to a method for determining the risk of bronchopulmonary dysplasia (BPD) of a preterm infant.

### BACKGROUND OF THE INVENTION

Preterm birth (PTB), or birth before 37 weeks of gestation period, is the major cause of neonatal mortality and morbidity worldwide. Approximately 70% of the neonatal deaths are due to preterm delivery [1]. Often defined as a complex, multifactorial syndrome, PTB can be categorized into “spontaneous” or “medically indicated” types. Seventy-percent of PTB cases are idiopathic, of which 45% is resulting from the spontaneous onset of labor (sPTB) and 25% from preterm premature rupture of membranes (PPROM). The remaining 30% are medically indicated preterm deliveries that are often preceded by complications including gestational diabetes mellitus (GDM), preeclampsia (PE), intrauterine growth restriction (IUGR), and infections such as chorioamnionitis [2]. Several mechanisms underlying PTB have been proposed, including stress-induced maternal and fetal hypothalamic-pituitary-adrenal (HPA) axis, genital infections and inflammation of decidua-amniochorion, decidual hemorrhage, or premature hormone change such as increased circulating CRH and relaxin levels [3]. However, the pathogenesis of preterm birth has not been fully unveiled. The current diagnostic methods of PTB include cervical length measurement (<30 mm) and fibronectin test in the cervicovaginal fluid, but the sensitivity and specificity of this method are both low, calling for identification of better biomarker that can render information on PTB risk [4].

Bronchopulmonary dysplasia (BPD), a common chronic inflammatory lung disease of very-low-birth-weight (VLBW) preterm infants, is associated with arrested lung development and treatment of supplemental oxygen [5]. Due to the influences of long-term oxygen therapy and mechanical ventilation, many of these preterm infants consequently acquire different types of problems, such as highly reactive airway diseases, recurrent lower respiratory tract infections, abrupt alveolar development, growth retardation, and feeding difficulties [6, 7]. While early detection of BPD is crucial to prevent chronic symptoms and complications later in life, diagnosis and prevention of this disease remains challenging due to the lack of good biomarkers for identification of infants at risk [5]. It has been reported that interleukin-8 (IL-8) and C-reactive protein (CRP), two recently identified preterm biomarkers, can also be biomarkers for BPD [8, 9]. Still, it is urgent to identify novel and efficient biomarkers for BPD.

The human erythropoietin-producing hepatocellular (Eph) receptors include transmembrane proteins comprising the largest family of receptor tyrosine kinases (RTKs). The first identified functions of Eph receptors were their roles in the complicated and sophisticated mechanism in axon guidance [10]. Eph receptors are now known to regulate a wide range of cell-to-cell communication events involved in cell positioning and tissue patterning during embryonic development and pathological conditions such as cancer and vascular complications [10-14]. In addition, these receptors are important regulators of specialized cell functions in synaptic plasticity, insulin secretion, bone remodeling, epithelial homeostasis, as well as inflammatory and immune responses [11, 12, 15]. They are expressed by a wide variety of cell types such as neurons, vascular cells, epithelial cells, inflammatory cells, immune cells, and tumor cells including cancer stem cells [16, 17].

EPHB2 gene belongs to the Eph receptor subfamily of the protein-tyrosine kinase family. Unlike ephrins-A, which are tethered to the plasma membrane via a glycosyl phosphatidyle inositol moiety, ephrins-B spans the plasma membrane and possess a short cytoplasmic tail. Previous studies have suggested EPHB2's functions in synaptic plasticity and neuronal process development, and the gene has recently been implicated in Alzheimer Disease pathology [18-20]. Moreover, recent studies have shown that variations in Eph and ephrin gene expression can be found in various types of human tumors such as carcinomas of the breast, lung, prostate, ovarian, neuroblastoma, and melanoma [21, 22].

A connection between EPHB2 and the TNF-α/NFκB signaling transduction have been reported recently in the brain [19]. Upon activation by TNF-α, trimerization of the TNF receptor occurs and results in recruitment of adapter proteins, leading to the release of NF-κB from IκB. NF-κB then translocates into the nucleus and initiates the transcription of EPHB2. The activated EPHB2 could promote cellular repair in damaged region in the case of injury or disease, but only if the surrounding environment is favorable for regrowth or survival; apoptosis could be the outcome otherwise [19].

### BRIEF SUMMARY OF THE INVENTION

The present invention provides a method for determining the risk of bronchopulmonary dysplasia (BPD) of a preterm infant, comprising detecting the expression level of EPHB2, in a primary culture of mesenchymal stem or stromal cells (MSCs) derived from a placenta-related tissue of the infant's mother or umbilical cord blood of the infant.

According to the present invention, the placenta-related tissue may be selected from the group consisting of amniotic membrane, chorionic disk, chorionic membrane, and umbilical cord.

According to the present invention, the expression level is a protein expression level or an mRNA expression level.

In certain embodiments of the present invention, the detecting step is performed through a polymerase chain reaction, real-time polymerase chain reaction, or quantitative polymerase chain reaction.

According to one embodiment of the present invention, the method further comprises the preliminary steps of collecting the MSCs from the placenta-related tissue, and culturing the MSCs in a culture medium to prepare the primary culture.

According to certain preferred embodiments of the present invention, the method further comprises a step of obtaining a ratio of the expression level of EPHB2 to a normal expression level of EPHB2. The normal expression level may an average expression level of EPHB2 in a plurality of primary cultures of MSCs derived from a plurality of placenta-related tissues of mothers of a plurality of full-term infants, or the full-term infants' umbilical cord blood.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the relative levels of IL-8 and EPHB2 transcripts determined by quantitative real-time polymerase chain reaction. The levels are relative to the average expression level of IL-8 or EPHB2 in samples in connection with full-term births. IL-8 is a known biomarker for BPD. Transcript levels of IL-8 and EPHB2 were determined in samples in connection with: (Group A—preterm birth with BPD) subjects Nos. TSG002, TSG008, LCG009, TSG010, LCG011, LCG012, LCG014 and LCG018; (Group B—preterm birth without BPD) subjects Nos. CK001, CK004, CK005, LCG006, CK013, TSG015, CK016 and CK017; and (Group C—full-term birth) subjects Nos. #006, #012, #017, #021, #023, #025, #026, #028, #061, #066, #067 and #075.

FIG. 2 shows the ratio of the gene expression (fold change) of EPHB2 and birth weight (kg). The EPHB2 expression level in samples in connection with preterm birth (with or without BPD) was divided by the average EPHB2 expression level in samples in connection with full-term birth. Then the ratio (fold change of expression level) was divided by the birth weight (kg) of a respective preterm infant.

FIG. 3 is an expression level—birth weight plot.

### DETAILED DESCRIPTION OF THE INVENTION

The present invention provides a method for determining the risk of developing bronchopulmonary dysplasia (BPD) of a preterm infant. The method comprises detecting the expression level of EPHB2 in a primary culture of mesenchymal stem or stromal cells (MSCs) derived from a placenta-related tissue of the infant's mother or umbilical cord blood of the preterm infant.

According to the present invention, the cells are freshly derived, obtained or collected from a placenta-related tissue following a protocol known in the art, for example, that of Shen et al [23]. The cells are then cultured in a culture medium for MSCs. A standard medium for MSC comprises Minimum Essential Medium Eagle (with different versions of modification), Fetal bovine serum (FBS) and basic fibroblast growth factor (bFGF).

The placenta-related tissue includes but is not limited to amniotic membrane, chorionic disk, chorionic membrane, or umbilical cord.

The primary culture of the cells derived from a placenta-related tissue is subjected to the detection of expression level of EPHB2.

As used herein, the term “expression level” refers to a protein expression level or an mRNA expression level.

In certain embodiments of the present invention, the detecting step is performed through a polymerase chain reaction, real-time polymerase chain reaction, or quantitative polymerase chain reaction. For example, the detection may be done by a real-time polymerase chain reaction instrument (LightCycler® 480 II, Roche).

According to one embodiment of the present invention, the method further comprises the preliminary steps of collecting the MSCs from the placenta-related tissue, and culturing the MSCs in a culture medium to prepare the primary culture.

According to certain preferred embodiments of the present invention, the method further comprises a step of obtaining a ratio of the expression level of EPHB2 to a normal expression level of EPHB2. The normal expression level may be an average expression level of EPHB2 in a plurality of primary cultures of MSCs derived from a plurality of placenta-related tissues of mothers of a plurality of full-term infants, or the full-term infants' umbilical cord blood.

According to certain embodiments of the present invention, the preterm infant is determined as at risk of BPD if Y<0.87X+0.16, where Y is the birth weight of the preterm infant in kilograms and X is the ratio of the expression level of EPHB2 to a normal expression level of EPHB2. In some other embodiments, the preterm infant is determined as at a lower risk of developing BPD if Y>0.87X+0.16, where Y is the birth weight of the preterm infant in kilograms and Xis the ratio of the expression level of EPHB2 to a normal expression level of EPHB2

The present invention is further illustrated by the following examples, which are provided for the purpose of demonstration rather than limitation.

### EXAMPLES

### Example 1: Quantitative Real-Time Polymerase Chain Reaction Evaluation of IL-8, EPHB2, NXPH2 and ST6GAL2 Transcript in Placenta-Derived Mesenchymal Stem Cells (MSCs)

Total RNA from 28 populations of placenta-derived cells (preterm birth, n=16 and full-term birth, n=12) were isolated using the Direct-zol miniprep Kit (Zymo Research Corporation, CA, USA). The complementary DNA (cDNA) was synthesized with Transcriptor First Strand cDNA Synthesis Kit (Roche, Basel, Switzerland). Then Quantitative RT-PCR was performed using the Roche SYBR Green System with a LightCycler480 II (Roche, Basel, Switzerland) according to the manufacturer's instructions.

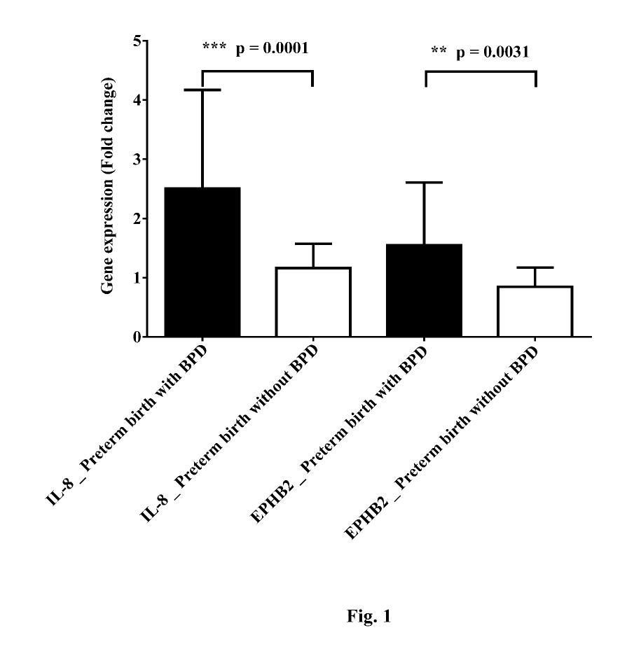

The expression levels of IL-8 and EPHB2 were determined by quantitative real-time polymerase chain reaction, wherein IL-8 is a known biomarker for BPD. Gene expression was normalized to the endogenous gene glyceraldehyde-3-phosphate dehydrogenase expression between different cell populations. The fold change is relative to the average expression level of IL-8 or EPHB2 in samples in connection with full-term births. As shown in FIG. 1, both IL-8 and EPHB2 expression levels were significantly different between the groups with and without BPD.

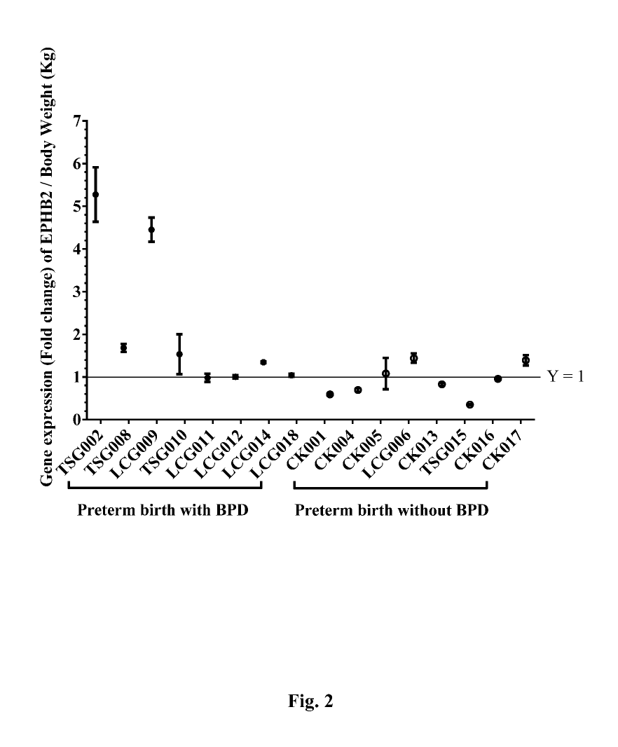

Subsequently, the relative expression level was divided by the birth weight of a respective preterm infant to obtain a ratio. The results are shown in FIG. 2. If the ratio of 1.0 is chosen to be the cut-off value, the results of risk determination are as in Table 1 below, with an accuracy higher than 80%.

TABLE 1
Risk determination for BPD

| | Preterm infants | Preterm infants |
|---|---|---|
| | with BPD | without BPD |
| Determined as BPD | 7 | 3 |
| Determined as non-BPD | 1 | 5 |

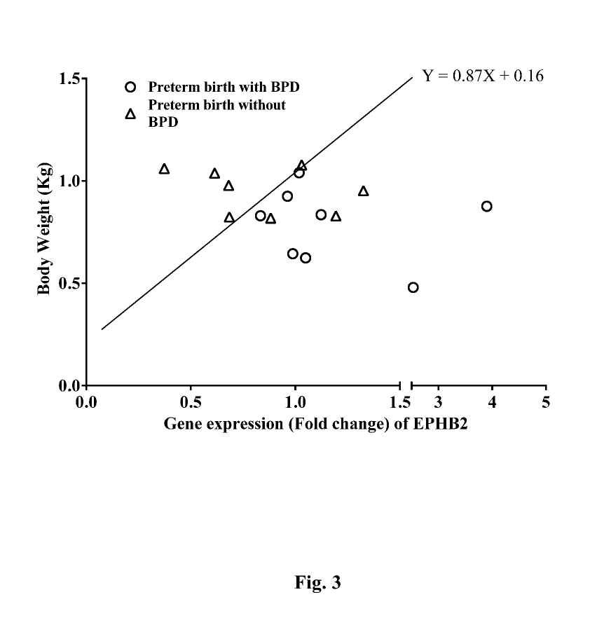

Further, as shown in FIG. 3, the preterm infant may be determined as at risk of BPD if Y<0.87X+0.16, and determined as at a lower risk of developing BPD if Y>0.87X+0.16, where Y is the birth weight of the preterm infant in kilograms and X is the ratio of the expression level of EPHB2 to a normal expression level of EPHB2.

### Example 2: Quantitative Real-Time Polymerase Chain Reaction Evaluation of EPHB2 Transcript in Umbilical Cord Blood (UCB)

Total RNA from umbilical cord blood (preterm birth, n=1 and full-term birth, n=6) was isolated using the Direct-zol miniprep Kit (Zymo Research Corporation, CA, USA). Subject No. CK020 is connection with a preterm birth without BPD. Subjects No. DM018, DM019, DM020, DM021, DM031 and DM032 are in connection with full-term births. The complementary DNA (cDNA) was synthesized with Transcriptor First Strand cDNA Synthesis Kit (Roche, Basel, Switzerland). Then Quantitative RT-PCR was performed using the Roche SYBR Green System with a LightCycler480 II (Roche, Basel, Switzerland) according to the manufacturer's instructions.

The EPHB2 expression in UCB was normalized to the endogenous gene glyceraldehyde-3-phosphate dehydrogenase expression. The fold change is relative to the average expression level of EPHB2 in samples in connection with full-term births. The results are shown in Table 2 below.

TABLE 2
Relative expression levels of EPHB2 in umbilical cord blood
(UCB; CK020).

| | Gene expression | |
|---|---|---|
| | (fold change) | |
| | of EPHB2 | Birth weight (kg) |
| CK020 | 0.778 | 1.098 |
| CK020 | 1.147 | 1.098 |
| CK020 | 0.952 | 1.098 |

Based on the results, it can be calculated that Y>0.87X+0.16, where Y is the birth weight of the preterm infant in kilograms and X is the ratio of the expression level of EPHB2 to a normal expression level of EPHB2.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### REFERENCES

[1] Shah J, Baxi B. Identification of biomarkers for prediction of preterm delivery. Journal of Medical society 2016:3-14.
[2] Eidem H R, Ackerman W Et, McGary K L, Abbot P, Rokas A. Gestational tissue transcriptomics in term and preterm human pregnancies: a systematic review and meta-analysis. BMC medical genomics 2015; 8:27.
[3] Kota S K, Gayatri K, Jammula S, Kota S K, Krishna S V, Meher L K, et al. Endocrinology of parturition. Indian journal of endocrinology and metabolism 2013; 17:50-9.
[4] Auray-Blais C, Raiche E, Gagnon R, Berthiaume M, J C. P. Metabolomics and preterm birth: What biomarkers in cervicovaginal secretions are predictive of high-risk pregnant women? Int J Mass Spectrom 2011; 307:33-8.
[5] Rivera L, Siddaiah R, Oji-Mmuo C, Silveyra G R, Silveyra P. Biomarkers for Bronchopulmonary Dysplasia in the Preterm Infant. Frontiers in pediatrics 2016; 4:33.
[6] Yang Y, Qiu J, Kan Q, Zhou X G, Zhou X Y. MicroRNA expression profiling studies on bronchopulmonary dysplasia: a systematic review and meta-analysis. Genetics and molecular research: GMR 2013; 12:5195-206.
[7] Maitre N L, Ballard R A, Ellenberg J H, Davis S D, Greenberg J M, Hamvas A, et al. Respiratory consequences of prematurity: evolution of a diagnosis and development of a comprehensive approach. Journal of perinatology: official journal of the California Perinatal Association 2015; 35:313-21.
[8] Paananen R, Husa A K, Vuolteenaho R, Herva R, Kaukola T, Hallman M. Blood cytokines during the perinatal period in very preterm infants: relationship of inflammatory response and bronchopulmonary dysplasia. The Journal of pediatrics 2009; 154:39-43 e3.
[9] Ambalavanan N, Carlo W A, D'Angio C T, McDonald S A, Das A, Schendel D, et al. Cytokines associated with bronchopulmonary dysplasia or death in extremely low birth weight infants. Pediatrics 2009; 123:1132-41.
[10] Wilkinson D G. Multiple roles of EPH receptors and ephrins in neural development. Nature reviews Neuroscience 2001; 2:155-64.
[11] Pasquale E B. Eph-ephrin bidirectional signaling in physiology and disease. Cell 2008; 133:38-52.
[12] Pasquale E B. Eph receptors and ephrins in cancer: bidirectional signalling and beyond. Nature reviews Cancer 2010; 10:165-80.
[13] Nievergall E, Lackmann M, Janes P W. Eph-dependent cell-cell adhesion and segregation in development and cancer. Cellular and molecular life sciences: CMLS 2012; 69:1813-42.
[14] Pitulescu M E, Adams R H. Eph/ephrin molecules—a hub for signaling and endocytosis. Genes & development 2010; 24:2480-92.
[15] Miao H, Wang B. Eph/ephrin signaling in epithelial development and homeostasis. The international journal of biochemistry & cell biology 2009; 41:762-70.
[16] Palmer A, Klein R. Multiple roles of ephrins in morphogenesis, neuronal networking, and brain function. Genes & development 2003; 17:1429-50.
[17] Hafner C, Schmitz G, Meyer S, Bataille F, Hau P, Langmann T, et al. Differential gene expression of Eph receptors and ephrins in benign human tissues and cancers. Clinical chemistry 2004; 50:490-9.
[18] Grunwald I C, Korte M, Wolfer D, Wilkinson G A, Unsicker K, Lipp H P, et al. Kinase-independent requirement of EPHB2 receptors in hippocampal synaptic plasticity. Neuron 2001; 32:1027-40.
[19] Paul D. Pozniak, Martyn K. White, Khalili K. TNF-α/NF-κB signaling in the CNS: possible connection to EPHB2. J Neuroimmune Pharmacol 2014 9:133-41.
[20] Cisse M, Halabisky B, Harris J, Devidze N, Dubal D B, Sun B, et al. Reversing EPHB2 depletion rescues cognitive functions in Alzheimer model. Nature 2011; 469:47-52.
[21] Tang X X, Evans A E, Zhao H, Cnaan A, Brodeur G M, N. I. Association among EPHB2, TrkA, and MYCN Expression in Low-stage Neuroblastomas. Med Pediat Oncol 2001; 36:80-2.
[22] Surawska H, Ma P C, Salgia R. The role of ephrins and Eph receptors in cancer. Cytokine & growth factor reviews 2004; 15:419-33.
[23] Shen S P, Liu W T, Lin Y, Li Y T, Chang C H, Chang F W, Wang L M, Teng S W, Hsuan Y. EphA2 is a biomarker of hMSCs derived from human placenta and umbilical cord. Taiwan J Obstet Gynecol. 2015; 54(6):749-56.

What is claimed is:

1. A method for determining the risk of bronchopulmonary dysplasia (BPD) of a preterm infant, comprising detecting the expression level of EPHB2 in a primary culture of mesenchymal stem or stromal cells (MSCs) derived from a placenta-related tissue of the infant's mother or umbilical cord blood of the infant.

2. The method of claim 1, wherein the placenta-related tissue is selected from the group consisting of amniotic membrane, chorionic disk, chorionic membrane, and umbilical cord.

3. The method of claim 1, wherein the expression level is a protein expression level or an mRNA expression level.

4. The method of claim 1, wherein the detecting step is performed through a polymerase chain reaction, real-time polymerase chain reaction, or quantitative polymerase chain reaction.

5. The method of claim 1, further comprising the preliminary steps of collecting the MSCs from the placenta-related tissue, and culturing the MSCs in a culture medium to prepare the primary culture.

6. The method of claim 1, further comprising a step of obtaining a ratio of the expression level of EPHB2 to a normal expression level of EPHB2.

7. The method of claim 6, wherein the normal expression level is an average expression level of EPHB2 in a plurality of primary cultures of MSCs derived from a plurality of full-term infants or their mothers.

8. The method of claim 6, wherein the preterm infant is determined as at risk of BPD if Y<0.87X+0.16, where Y is the birth weight of the preterm infant in kilograms and X is the ratio of the expression level of EPHB2 to a normal expression level of EPHB2.
